(19) United States
(12) Patent Application Publication
(10) Pub. No.: US 2014/0013574 A1
(43) Pub. Date: Jan. 16, 2014

(54) SYSTEMS AND METHODS FOR ATTACHING RADIOPAQUE MARKERS TO A MEDICAL DEVICE

(71) Applicant: Intact Vascular, Inc., Wayne, PA (US)

(72) Inventor: Robert M. Giasolli, Orange, CA (US)

(21) Appl. No.: 13/939,019

(22) Filed: Jul. 10, 2013

Related U.S. Application Data
(60) Provisional application No. 61/670,570, filed on Jul. 11, 2012.

(51) Int. Cl. A61B 19/00

(52) U.S. Cl. CPC A61B 19/54
USPC 29/505; 29/56.5

(57) ABSTRACT

A radiopaque marker can be attached to a medical device, such as an intravascular device. The radiopaque marker can be pre-formed, or formed from a spool of material. The radiopaque marker can be placed in, on, or at a spot for receiving the radiopaque marker. The radiopaque marker can then be attached to the medical device.

### CROSS-REFERENCE TO RELATED APPLICATIONS

This application claims priority to U.S. Provisional Appl. No. 61/670,570 filed Jul. 11, 2012. The entire contents of the above applications are hereby incorporated by reference and made a part of this specification. Any and all priority claims identified in the Application Data Sheet, or any correction thereto, are hereby incorporated by reference under 37 CFR 1.57.

### BACKGROUND

1. Field of the Invention

Certain embodiments disclosed herein relate generally to attaching materials to medical devices. In particular, the materials can be used as radiopaque markers. The medical devices can include intravascular devices (such as stents, tacks, staples) and other medical devices and/or tools especially where it may be desirable to view the device or tool under radiographic imaging.

2. Description of the Related Art

In modern medicine, radiodense substances are those that will not allow X-rays or similar radiation to pass. Medical devices often contain a radiopaque marker to enhance visualization during implantation and/or for monitoring the position of the medical device.

### SUMMARY OF THE INVENTION

There exists a constant need for improvement in systems and methods for attaching radiopaque materials to medical devices. A radiopaque marker can be placed in, on, or at a spot for receiving the radiopaque marker on a medical device. Energy, such as a force, can be applied to the radiopaque marker to attach the radiopaque marker to the device. The radiopaque marker can be pre-formed, or can be formed from a spool of material.

In some embodiments, a method of attaching a radiopaque marker to a medical device can comprise one or more of the following steps. Picking up a radiopaque marker. Positioning the radiopaque marker at a designated location on a medical device. Applying energy to the radiopaque marker to connect the radiopaque marker to the medical device at the designated location.

In certain embodiments, a method of attaching a radiopaque marker to an intravascular device can comprise one or more of the following steps. Applying suction to a radiopaque marker. Positioning the radiopaque marker above a receiving hole in an intravascular device. Releasing suction from the radiopaque marker such that the radiopaque marker is placed on the receiving hole. Applying a force on the radiopaque marker to deform the radiopaque marker such that the radiopaque marker connects to the receiving hole. In some embodiments, the method may also include a first head forming at a first end of the radiopaque marker and a second head forming at the opposite end.

According to some embodiments, a method of attaching a radiopaque marker to an intravascular device can comprise one or more of the following steps. Positioning an end of a wire in a wire dispensing tool head above a receiving hole in an intravascular device. Placing the end of the wire on or in the receiving hole. Cutting the wire. Applying a force on the cut wire to deform the cut wire such that the cut wire forms a radiopaque marker connected to the receiving hole.

According to some embodiments, a system can be used for attaching a radiopaque marker to an intravascular device. The system can include a wire dispensing tool, a cutting tool, and a hammer tool. The wire dispensing tool can be configured to precisely position an end of a wire at a receiving hole in an intravascular device. The cutting tool can be configured to cut off the end of the wire after it is positioned at the receiving hole. The hammer tool can be configured to apply a force on the cut wire end to deform the wire end such that the wire end connects to the receiving hole.

According to some embodiments, a method of attaching a radiopaque marker to an intravascular device can comprise one or more of the following steps. Advancing a wire such that a wire tip extends past a wire dispensing tool head. Applying an electric discharge to the wire tip, thereby forming a ball. Positioning the ball at a receiving hole in an intravascular device. Cutting the wire. Applying a force on the ball to deform the ball such that the ball forms a radiopaque marker connected to the receiving hole.

### DRAWINGS

Various embodiments are depicted in the accompanying drawings for illustrative purposes, and should in no way be interpreted as limiting the scope of the inventions. In the drawings, like reference characters denote corresponding features consistently throughout similar embodiments.

FIG. 1 is a perspective view of a positioning machine.

FIGS. 2A-B show schematic views of a positioning machine head in two different positions.

FIG. 3 is a flow chart outlining certain steps of a method for connecting a radiopaque marker to a medical device.

FIG. 4A illustrates a schematic view of a holding piece with an intravascular device positioned thereon, a plurality of radiopaque markers and the positioning machine head of FIG. 2A.

FIGS. 4A-F show various steps in a method for connecting a radiopaque marker to an intravascular device.

FIG. 5 is a detail view of a radiopaque marker in a hole of a medical device.

FIG. 6A shows a spool of material for making a radiopaque marker.

FIGS. 6B-C illustrate various steps in a method for connecting a wire based radiopaque marker to an intravascular device.

FIGS. 7A-B show various steps in a method for forming a wire based radiopaque marker.

FIG. 7C is a step in a method for connecting a wire based radiopaque marker to an intravascular device.

FIG. 8A-B show various steps in another method for forming a wire based radiopaque marker.

FIG. 9 shows a detail view of a cutting tool.

FIG. 10 shows a detail view of another cutting tool.

### DESCRIPTION OF CERTAIN EMBODIMENTS

Radiopaque markers are commonly used on many different types of medical devices. Methods and systems of attaching radiopaque markers to intravascular devices, such as stents, staples and tacks are described below. However, it should be understood that the principles of the systems and methods described herein can be employed for attaching materials other than radiopaque markers as well as for medical devices other than intravascular devices, including, but not limited to, delivery devices, tools, implants, etc.

A radiopaque marker can be attached to a medical device, such as an intravascular device. The radiopaque marker can be pre-formed, or formed from a spool of material. The radiopaque marker can be placed in, on, or at a spot for receiving the radiopaque marker on the medical device. Energy, such as a force, can be applied to the radiopaque marker to attach the radiopaque marker to the intravascular device.

Pick And Place

In certain embodiments, a pick and place method of attaching radiopaque markers to a medical device can be used. The medical device can include a hole, slot, indention, or other space for receiving the radiopaque marker. A manual or automated process can be used to pick up the marker from a first holding piece. This can be done with a grabber, such as a tool head with a vacuum pickup tool. The marker can then be placed at or in the space designated for the radiopaque marker. Once placed, the radiopaque marker can be released. This can be done, for example, by releasing the suction on the vacuum pickup tool. Energy, such as a force, can then be applied to the radiopaque marker, to force the radiopaque marker into the space designated for the radiopaque marker. This may be done with the same tool, or a separate tool, such as a hammer and/or riveter tool head.

As radiopaque markers are often made with gold or other malleable materials, applying a force on the radiopaque marker can cause the radiopaque marker to deform. The radiopaque marker and device can connect by forming a friction fit within the space designated for the radiopaque marker, but it may also connect by forming a molecular bond between the radiopaque marker and the device. Other types of connections are also possible, some examples of which are described elsewhere herein. Where the space designated for the marker is a hole, a head on one or both ends of the marker may be formed. The heads can be on either side of the hole.

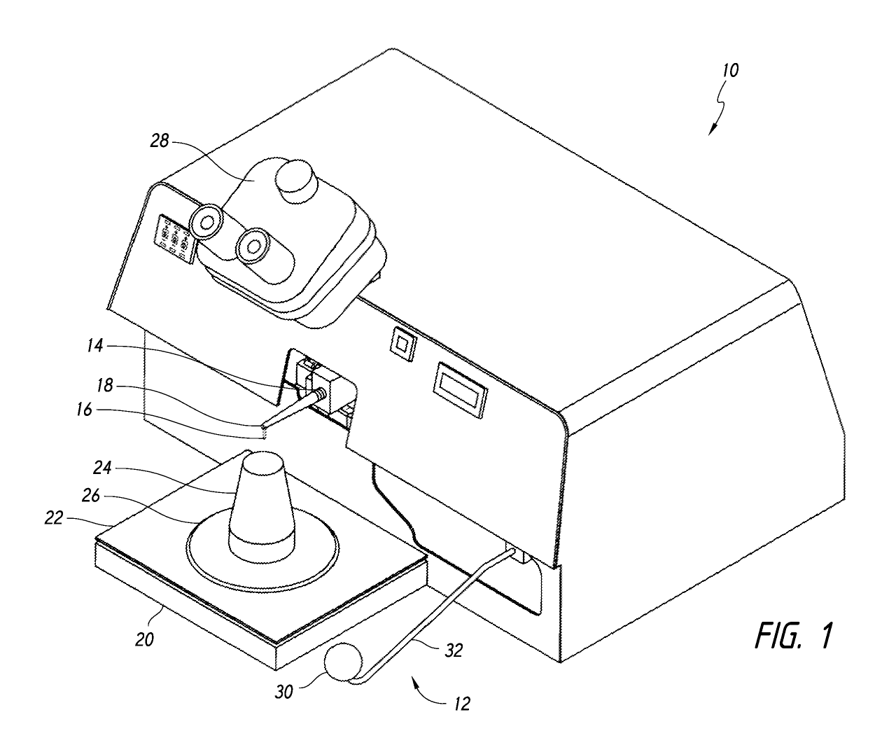

A positioning device, such as a micropositioner apparatus, can be used in some methods to place the radiopaque marker on the medical device and/or connect it to the medical device. FIG. 1 illustrates a manual micropositioner apparatus 10 according to some embodiments. A micropositioner apparatus 10 can have a manually operable input mechanism 12 coupled to a tool head 14. The motions of the input mechanism 12 can cause precise corresponding motions to follow at the tool head 14. The micropositioner apparatus 10 can include a pantograph-like input manipulator mechanism to precisely move a tip 16 of a tool 18 connected to the tool head 14. The tool head 14 can be made to move in coordinate directions in precisely scaled fractions of corresponding motions of the input mechanism 12.

The micropositioner apparatus 10 can be used to precisely position small radiopaque markers at precise locations on a medical device. Many medical devices, such as stents, staples, and tacks are fairly small and have very little space to attach additional materials. Stents for example, are often skeletons that include only the bare minimum amount of material. In addition, radiopaque materials can be expensive compared to the cost of materials for the rest of the device and so it can be desirable to have a very small radiopaque marker.

Continuing to look to FIG. 1, the micropositioner 10 provides the ability to precisely move the tip 16 of the tool head 14 relative to a miniature workpiece such as a stent. The micropositioner 10 can include a base plate 20 which supports a work table 22. A holding piece 24 can be positioned on the work table 22. The holding piece 24 can be designed to hold one or more medical devices to allow connection of one or more radiopaque markers to the medical device(s). The holding piece 24 can be stationary or movable. For example, the illustrated holding piece 24 also includes a turntable 26. Example holding pieces 24 can include, but are not limited to a pedestal, a vice and a collet. In addition, one or more holding piece can be used. For example, a plurality of radiopaque markers can be placed on a first holding piece, such as a pedestal, and a stent can be positioned around a second holding piece in the form of a rod.

It will be understood that the holding piece 24, work table 22 and base plate 20 can include any number of different configurations. In some embodiments, the work table 22 includes railings and/or is magnetic to allow different types of holding pieces to be easily connected or disconnected thereto. The work table itself may also be adjustable in one, two, or three different axes. In some embodiments, the position of the work table can be manually and/or automatically controlled. It will be understood that the work table 22 can be configured and can function in ways similar to other known manufacturing tools, such as the work table of a milling machine or lathe.

A viewing system 28 can be provided with the micropositioner 10. For example, a stereoscopic microscope 28, or a camera and video screen can be used to magnify an image of the workpiece and tool head to facilitate precise placement of the radiopaque markers.

The input mechanism 12 can include any number of different mechanisms. For example, the input mechanism 12 can include a control knob 30 attached to the outer end of a control arm 32. Other input mechanisms can include mechanical and/or electronic controls such as one or more joystick, sensor, button, lever, computer mouse, touchpad, etc.

In some embodiments, the input mechanism 12 includes a longitudinally outwardly protruding control arm 32 having a control knob 30 at the outer longitudinal end thereof, for grasping between the thumb and fingers of a human operator. The control arm can be coupled to the outer lateral end of a four-bar parallelogram linkage comprising part of a pantograph mechanism, while the inner lateral end of the pantograph mechanism can be pivotably connected through vertically and horizontally disposed pivot support bearings to a support structure. Motions of the control knob 30 can be transferred to the tool head 14 through the pantograph mechanism. The pantograph mechanism can be used to move the tool head 14 in precisely scaled fractions of corresponding movements of the control knob 30.

Manual die bonder machines are example micropositioner apparatus that can be used and/or modified to perform certain of the methods described herein. An example manual die bonder machine is a dual head epoxy die bonder machine (model number 7200CR), available from West Bond, Inc. of Anaheim, Calif. Additional examples of micropositioner apparatus that can be used and/or modified to perform certain of the methods described herein can be found in U.S. Pat. No. 5,871,136, entitled “Micropositioner for Ultrasonic Bonding” and incorporated herein by reference in its entirety.

Various different types of tool heads can be used with a micropositioner. For example, a single tool head, a dual tool head, or an interchangeable tool head could be used. In addition, the tool head can include a number of tool heads connected in parallel so that each head can work simultaneously with a separate medical device and/or a separate portion of a medical device.

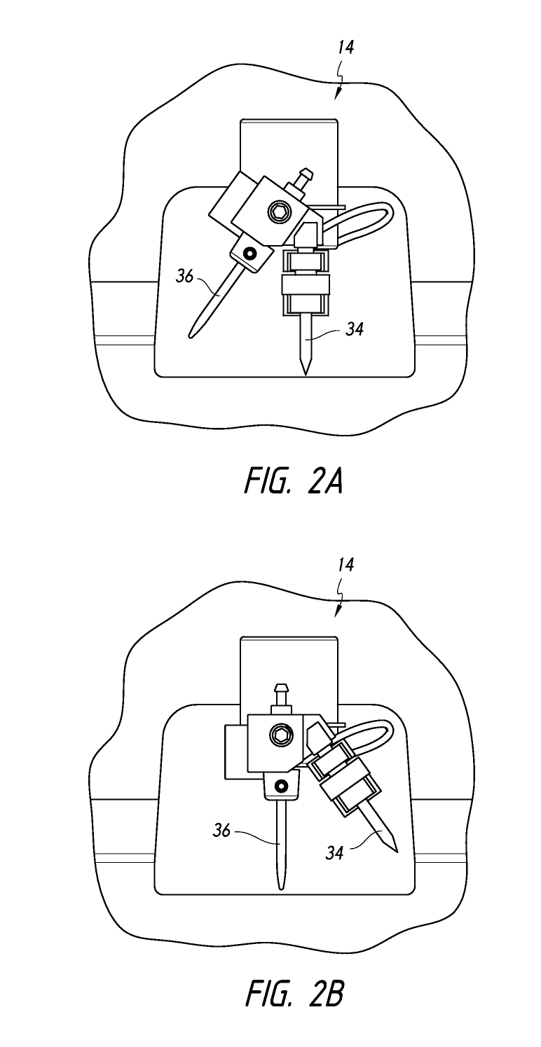

Turning now to FIGS. 2A-B, schematic views of a tool head 14 in two different positions are shown. A dual tool head 14 is illustrated in FIG. 2A in a first position configured for use of a first tool 34. FIG. 2B shows the dual tool head 14 in a second position configured for use of a second tool 36. The dual tool head 14 can be switched between tool heads for use of the different tools.

For example, with a manual micropositioner 10, moving the control knob 30 upwards to a set point (which also moves the tool head) can switch the tool head between two or more different tools.

In some embodiments, such as that shown in FIGS. 2A-B, one of the tool heads can be a vacuum pickup tool 34, or other type of grabbing tool. The vacuum pickup tool 34 can be used to pick up a radiopaque marker, such as a gold ball or bar. The radiopaque marker can then be placed on the medical device. The vacuum can be turned off to release the radiopaque marker at the desired location. The dual head 14 can then be switched between tool heads.

The second head can be a hammer 36, riveter, or other tool that can attach the radiopaque marker to the medical device at the desired location. In some embodiments, the head can be a spot welder or other device to impart energy (mechanical, electrical, optical, microwave, acoustic, ultrasonic, IR, arc voltage, etc.) on the marker. As shown, the second head 36 is a bar or tip that can function as a hammer and/or riveter. The second head can force the marker into a hole in the medical device. In these embodiments the holding piece 24 may also serve as a type of anvil or forging tool. The radiopaque marker can be malleable such that the force imparted by the hammer deforms the marker. This can cause the radiopaque marker to expand in one direction, while contracting in another direction. This expansion can also cause the radiopaque marker to connect to the medical device.

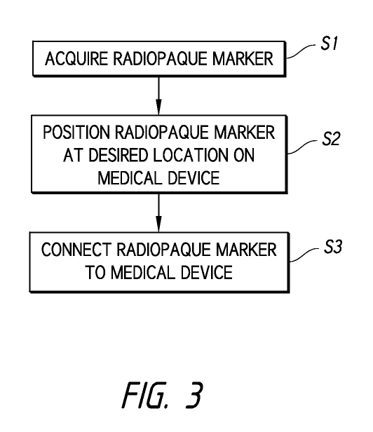

Moving now to FIG. 3, a flow chart illustrates basic steps of certain embodiments of a pick and place method for attaching a radiopaque marker on a medical device. At step one S1, a tool head acquires a radiopaque marker. At step two S2, the radiopaque marker is positioned at a desired location on the medical device. Then, in step three S3 the radiopaque marker is connected to the medical device.

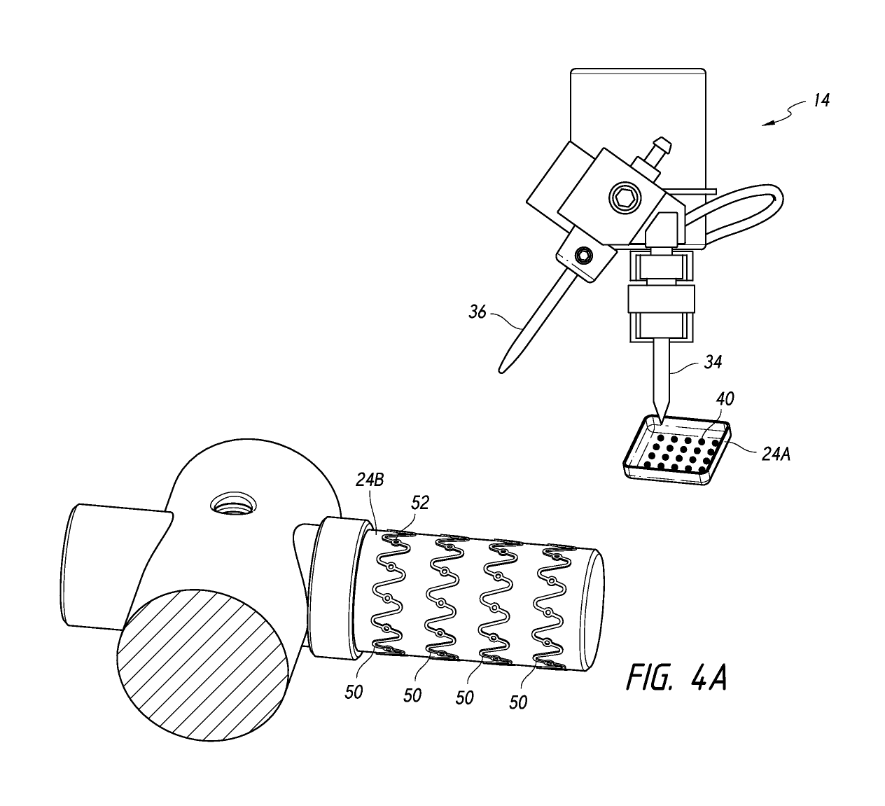

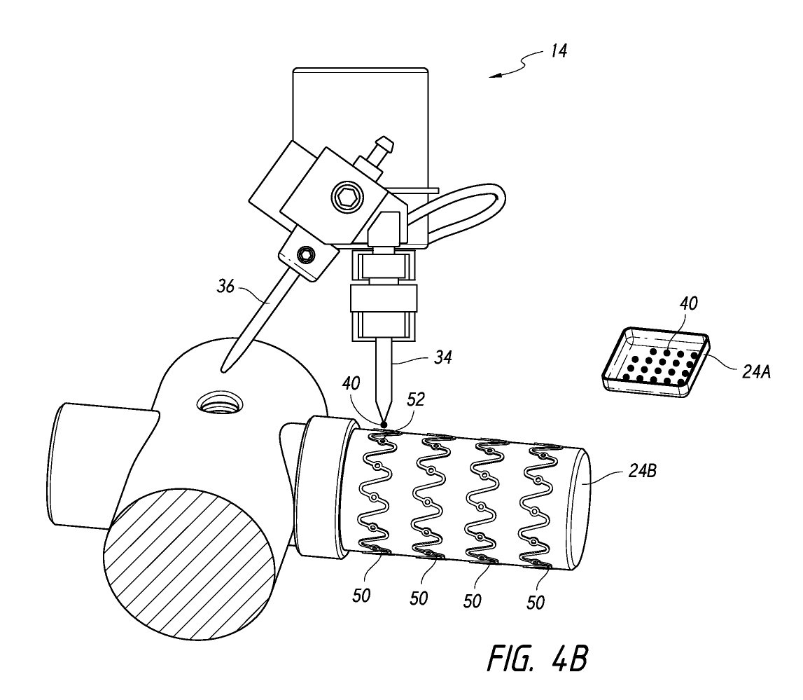

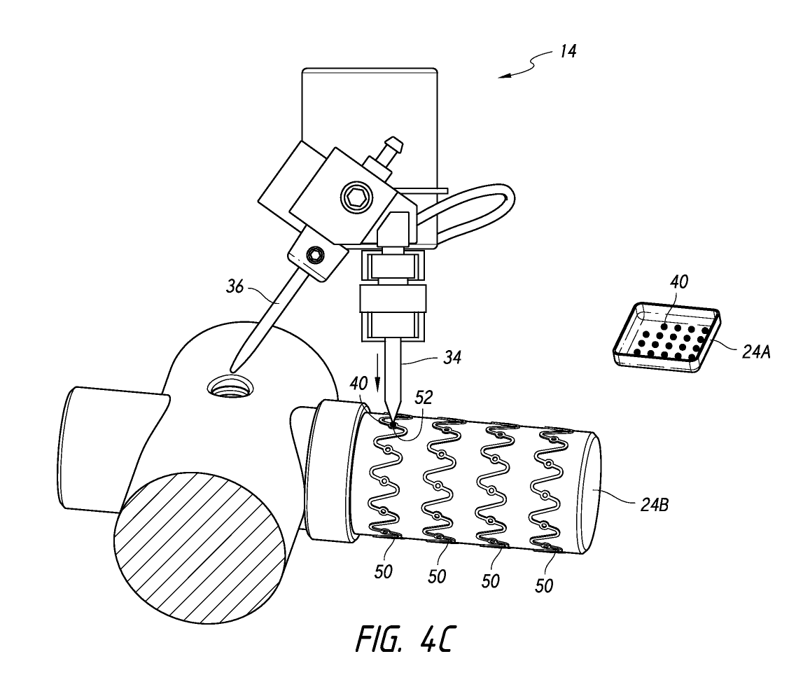

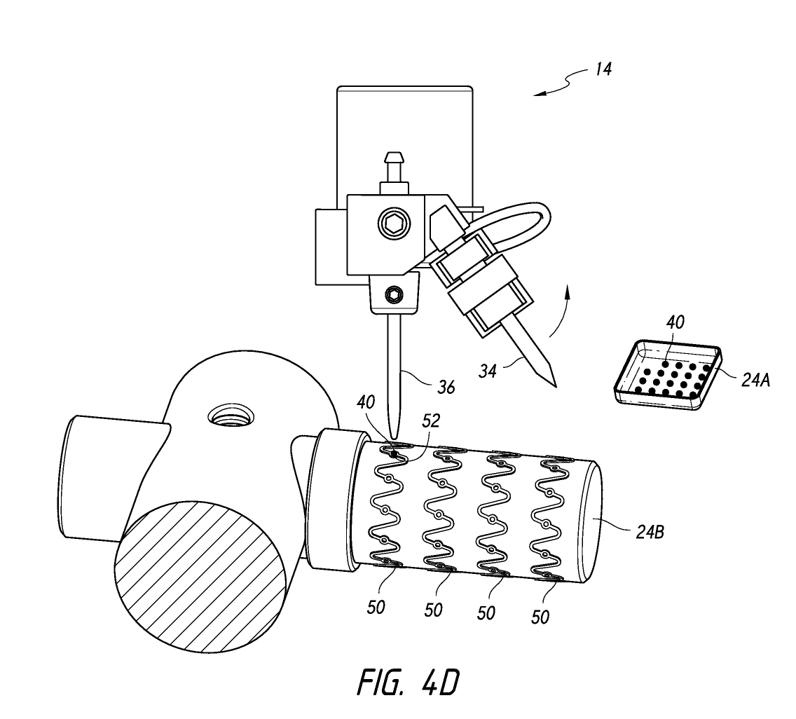

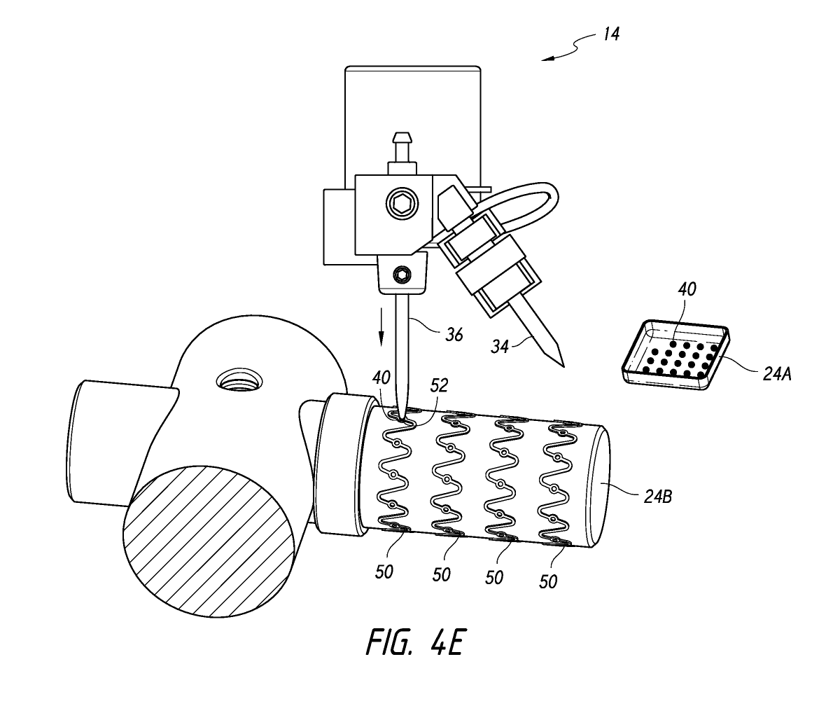

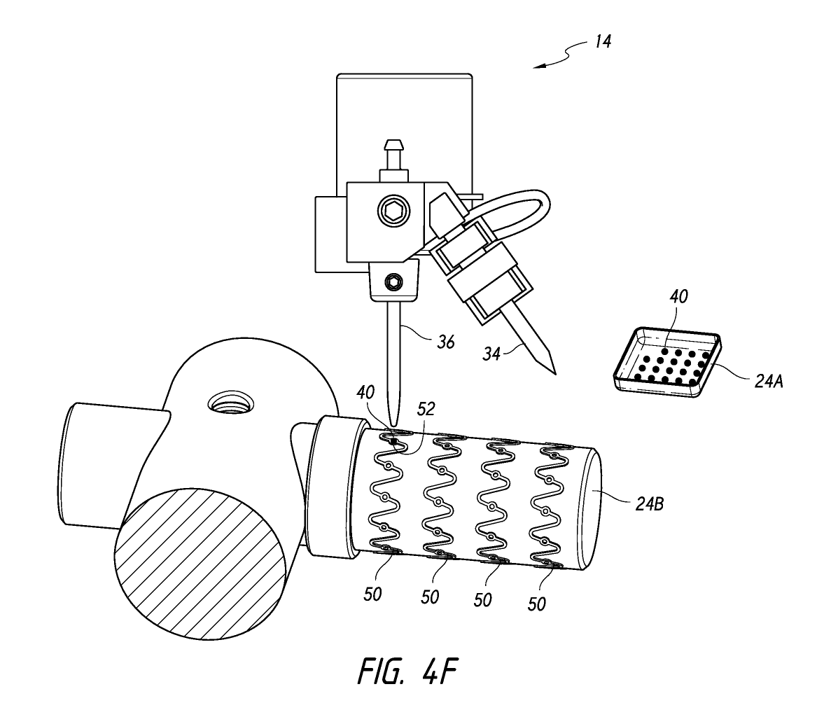

FIGS. 4A-4F show certain steps in a method for connecting a radiopaque marker to an intravascular device. In some embodiments, the intravascular device can be the tack device described in U.S. Patent publications 2012/0035705 and 2013/0144375, both of which are incorporated herein by reference and are to be considered part of this specification. The intravascular device can be the tack device, but can also be a stent or staple. The method can also be practiced on other medical devices, especially a device designed for use within the body and typically placed under angioscopic guidance.

As shown in FIG. 4A, a tool head 14 including a vacuum tool 34 or other grabber can be positioned above a radiopaque maker 40. The radiopaque marker 40 can be positioned together with one or more additional radiopaque markers on a first holding piece 24A. The radiopaque markers are shown spaced apart to simplify a pick and place procedure. In some embodiments, the first holding piece 24A can include a plate or a backing material. The plate can be on a pedestal or be part of a pedestal.

One or more intravascular device(s) 50 can be positioned on a second holding piece 24B. The illustrated embodiment shows a holding piece 24B with four equally spaced intravascular devices 50 and a separate holding piece 24A with a number of radiopaque markers 40. The illustrated second holding piece 24B is formed of a metal rod. This can allow the holding piece to also serve as an anvil as will be described in more detail below.

As an initial step, the vacuum tool 34 can pick-up one of the radiopaque markers 40. It will be understood that other grabbing or picking tools could alternatively be used at this step. In some embodiments, a plurality of radiopaque markers can be positioned within a magazine or on a movable base. This can allow the system to repeatably place a radiopaque marker at the same location for pick up, after a first radiopaque marker is picked up by the vacuum tool or other grabber type tool. In an alternative embodiment, a magazine loaded with radiopaque markers can be part of a tool head.

After acquiring the radiopaque marker 40, the tool head 14 can be moved to position the radiopaque marker at a desired location 52 on the intravascular device 50 (FIG. 4B). The desired location can be a hole, slot, indention, or other space for receiving the radiopaque marker. The illustrated intravascular devices 50 include a plurality of holes 52 for receiving the radiopaque marker 40 spaced around the intravascular device 50.

Once the radiopaque marker 40 is properly positioned at the desired location 52 of the intravascular device 50, the radiopaque marker 40 can be released, placing it on or in the desired location 52 (FIG. 4C). For example, in the illustrated embodiment, the vacuum tool 34 positions the radiopaque marker 40 at or above the hole 52 in the intravascular device 50 (FIG. 4B). The vacuum can then be turned off. This can release the radiopaque marker 40, placing it on or in the hole 52 (FIG. 4C).

With the radiopaque marker 40 properly positioned at the desired location 52 it can be attached to the intravascular device 50. It will be understood that the attachment step may be performed before or after release of the radiopaque marker from the tool head. The radiopaque marker 40 can be connected to the medical device in one of many different manners; for example, friction fit, welding, adhesive, bonding, friction, screw, nail, shaping, riveting, molecular bonding, etc. In some embodiments, a force can be applied to the radiopaque marker 40 to attach the radiopaque marker to the medical device 50.

One manner in which the radiopaque marker 40 can be attached to the intravascular device 50 is through deformation. As has been mentioned, the tool head can be a dual tool head, with a vacuum tool 34 and the hammer tool 36. Where this is the case, after the radiopaque marker 40 has been released, the tool head can be changed so that the hammer 36 can be in position for use. In the device shown, the tool head can automatically switch by moving the tool head upwards until it reaches a set height. This action can auto switch the tool head between tools. FIG. 4D shows the tool head 14 in the second position with the hammer 36 ready for use.

The second head of the tool head 14 can be a hammer 36 to deform and/or force the radiopaque marker 40 into connection with the medical device at the desired location. In the illustrated embodiment, the hammer 36 can force the radiopaque marker 40 into the hole 52 in the intravascular device 50. The radiopaque marker 40 can be made of a malleable material such as gold so that the second holding piece 24B can serve as an anvil. The second holding piece 24B can constrain movement of the radiopaque marker 40 at one end of the hole 52 as the hammer 36 drives the radiopaque marker 40 into the other end. FIG. 4E shows the hammer pressing the radiopaque marker 40 into the hole 52. This movement can be a slow press or a quick strike. The hammer head mechanism can beneficially achieve repeatable and accurate presses or strikes at the point where the hammer hits the radiopaque marker. In some embodiments, the hammer can apply a relatively high force that equals or exceeds about 1000 mg (0.1N). In some embodiments, the hammer can apply a relatively high force that equals or exceeds about 0.5, 1, 1.5, 2, 3, 4, or 5N. In some embodiments, the hammer can apply a force of between about: 1N to 30N, 5N to 20N and 5N to 10N.

The radiopaque marker 40 can be malleable such that the force imparted by the hammer 36 deforms the marker. This may cause the radiopaque marker 40 to expand in one direction, while contracting in another direction. This expansion can also cause the radiopaque marker 40 to connect to the intravascular device 50. In some embodiments, deforming the radiopaque marker 40 can force it to expand within the hole, and force one or more of the top and bottom to expand to form a head on either or both of the top and the bottom of the marker. FIG. 4F shows the flattened radiopaque marker 40 connected to the intravascular device 50.

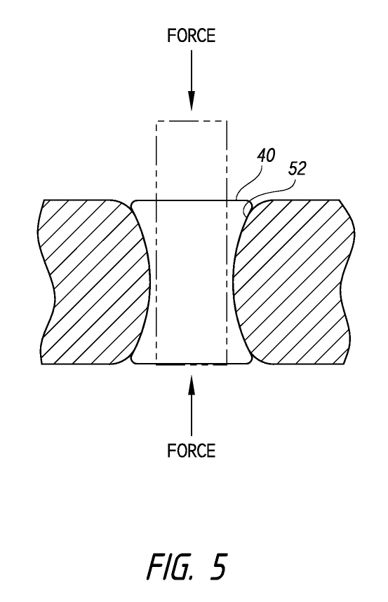

Looking now to FIG. 5, a schematic representation of a bar shaped radiopaque marker 40 is shown within a hole 52 in a medical device. Forces can be applied on either side of the device to deform the radiopaque marker into a sort of hour-glass or barbell shape. For example, the marker can be smashed between the holding piece and a hammer tool. In some embodiments the holding piece can move upward as the hammer is pressed down. This can allow for greater control of the expansion of the radiopaque marker 40 within the hole 52. By applying pressure from both sides the center bulges out first and minimizes any cavity formation.

The deforming action may cause a head to form on the marker at one or both ends. Thus, the marker head(s) can be on either side of the hole. In some embodiments, deforming the marker can cause it to take a shorter and stouter shape. The deforming action provides shaping of the marker against the sides or walls of the hole. The marker volume can be equal to or slightly greater than the volume of the hole. This can help ensure that the radiopaque marker fits within and fills the hole without gaps or spaces between the marker and the sides or walls of the hole.

In some embodiments, a method of attaching a radiopaque marker to a medical device can include A) grabbing a radiopaque marker; B) positioning it over a hole; C) releasing it in the hole; and D) pressing down on the radiopaque marker in the hole so that the radiopaque marker deforms and is connected to the medical device.

A method of attaching a radiopaque marker to an intravascular device can comprise: applying suction to a radiopaque marker; positioning the radiopaque marker above a receiving hole in the intravascular device; releasing suction from the radiopaque marker such that the radiopaque marker is placed on the receiving hole; and applying a force on the radiopaque marker to deform the radiopaque marker such that the marker connects to the receiving hole. This may also cause a first head forming at a first end of the radiopaque marker and a second head forming at the opposite end.

Once the radiopaque marker 40 has been connected to the medical device 50 the procedure can be repeated to connect a second radiopaque marker 40 to a medical device 50. The medical device 50 may be the same device or different device. In the example shown, four intravascular devices 50 are lined up next to each other. Thus, an operator after attaching the first radiopaque marker 40 can then attach a second radiopaque marker 40 to either the same intravascular device 50 at a different location, or to a different device.

In some embodiments, the holding piece 24 can be rotated, advanced, or otherwise moved to adjust the position of the medical device(s). Alternatively, or in addition, the tool head 14 can be used to move the medical device(s). The medical device(s) in the new position can allow connection of another radiopaque marker 40 to the medical device(s).

For example, the hammer 36 can be used to advance the intravascular device 50 on the holding piece 24B. This can position the next hole to receive a radiopaque marker. The hammer 36 can push the intravascular device 50 up or down around the rod so that the next hole will be in position. Once repositioned, a new marker can be placed in the next hole in the same manner as previously discussed.

The illustrated radiopaque marker 40 is in the shape of a ball, though it will be understood that the marker can be formed in any number of shapes. The shape of the marker can, among other things, be a factor of the location for receipt in the medical device, the desired size, the connecting mechanism etc. Example shapes for the radiopaque marker 40 include ball, disk, cylinder, disk or column with concave sides, cube, etc. The marker volume can be equal to or slightly greater than the volume of the hole. This can help ensure that the marker fits within and fills the hole without gaps or spaces between the marker and the sides or walls surrounding the hole. In addition, where the marker is a ball, the ball diameter can be greater than the diameter of the hole. Radiopaque markers can be made of any of a number of different materials including, but not limited to, titanium, tungsten, and gold.

Spool Delivery

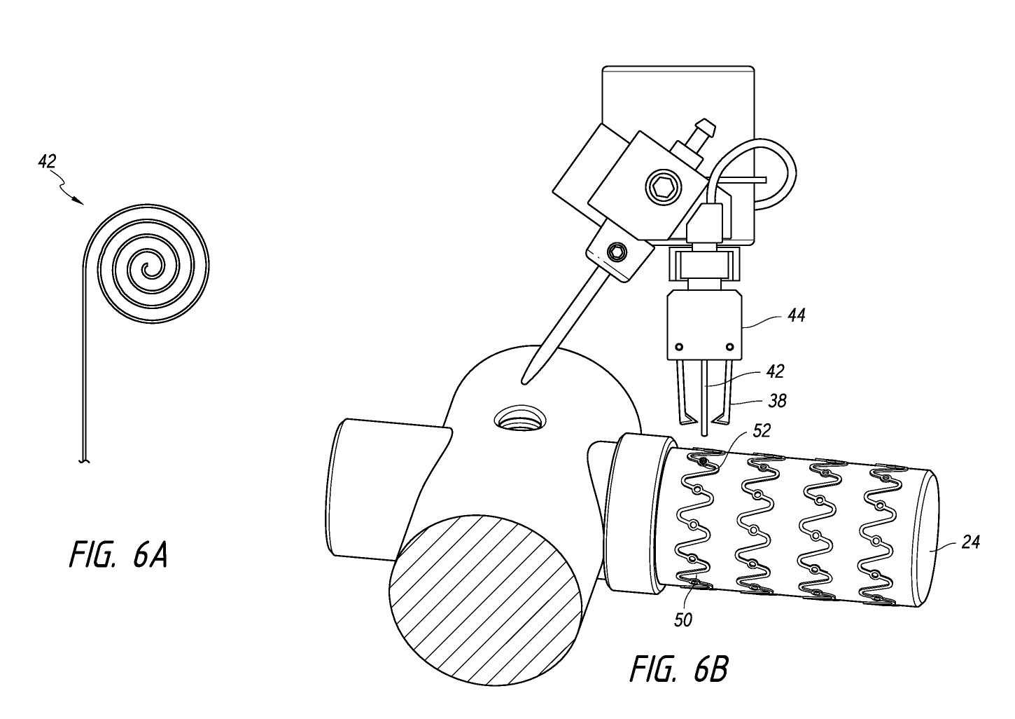

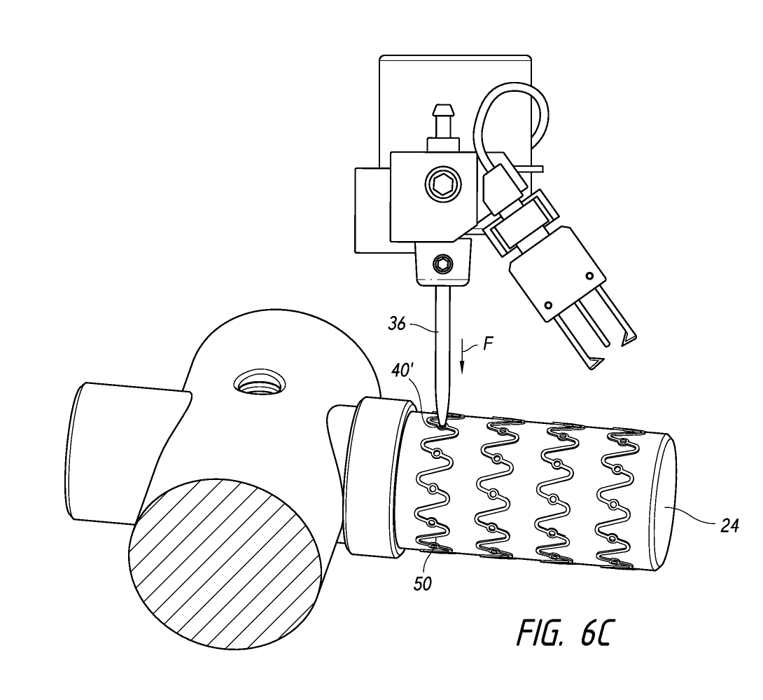

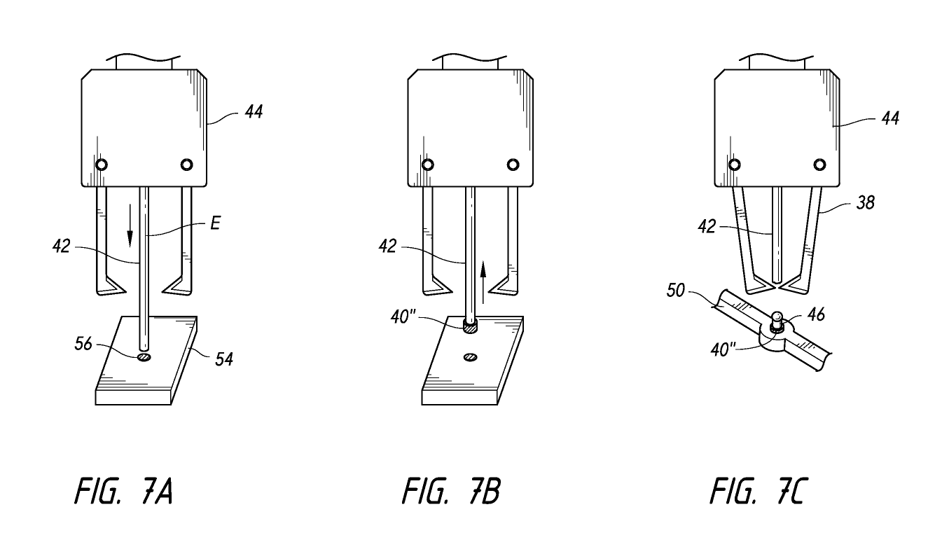

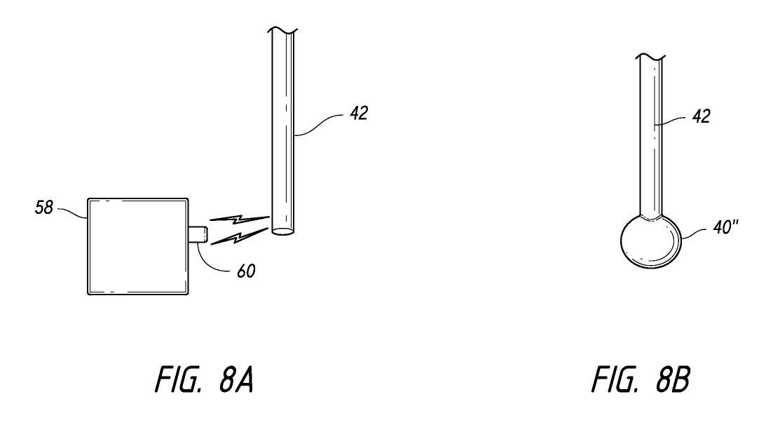

In some embodiments, a material on a spool, such as metal wire or ribbon 42, can be used to form the radiopaque markers (FIG. 6A). An end of the wire 42 to be used as a radiopaque marker can be positioned at the desired location 52 on the medical device. As before, the desired location can be a hole, slot, indention, or other space for receiving the radiopaque marker. The wire 42 can then be cut. Energy, such as a force, can be applied to the cut wire 40′ to deform the wire to form a secure attachment between the wire based radiopaque marker 40′ and the medical device. Similar to the pick and place method, a head may be formed on one or both ends of the wire based radiopaque marker 40′.

Looking now to FIG. 6B, it can be seen that the wire 42 may be positioned at a hole 52. The wire can then be moved to be within the hole or it may be positioned over the hole, such that when cut, the radiopaque marker 40′ will be positioned within the hole 52. It will be understood that in other embodiments, the wire 42 can be cut and then the wire based radiopaque marker 40′ can be placed and/or positioned at the desired location 52.

A cutting tool, or cutting head 38 as shown in FIG. 6B can be used to cut the material to the desired size for a radiopaque marker. The cutting tool 38 can have any number of different configurations. The illustrated embodiment includes two opposing blades, each extending at an angle from an arm member. The arm members can be moved closer together to create the cutting motion.

A material handling or wire dispensing tool 44 can be used to control the position of the wire and the amount of wire that in unwound from the spool. After the wire 42 has been cut to form a radiopaque marker 40′, the material handling tool 44 can advance the wire a predetermined amount to prepare the wire for the next cutting. The cutting tool 38 and material handling tool 44 can be part of the same or different tool heads.

As can be seen in FIG. 6C, with the wire based radiopaque marker 40′ positioned at the desired location 52, a force can be applied to the radiopaque marker 40′ to connect the radiopaque marker 40′ to the medical device. A hammer tool 36 can be used to apply the force. Similar to the pick and place method, a head may be formed on one or both ends of the wire based radiopaque marker 40′. The radiopaque marker 40′ can be connected to the medical device in many other ways as well, as have been described above (for example, friction fit, welding, adhesive, bonding, friction, screw, nail, shaping, riveting, etc.).

Similar to the above description related to a pick and place method, a micropositioner apparatus 10 can be used to precisely position wire based radiopaque markers 40′ at precise locations on a medical device. In addition, the micropositioner apparatus 10 can be used to create radiopaque markers 40′ from a spool of material such as metal wire or ribbon.

Similar to the pick and place description, a bonder machine, available from West Bond, Inc. of Anaheim, Calif. can be modified to perform the method. In this case, the bonder machine can be a wire bonder machine, such as a series 7400E, 7600E or 7700E wire bonder.

As has been mentioned, rather than using preformed markers, such as balls or bars, a spool of wire or ribbon can be used. The size or gauge of the wire, as well as other dimensions and/or shape of the wire can be a factor of the size hole or location for receipt of the ultimate radiopaque marker 40′. The shape of a round, flat, or other shaped wire and length to be cut can be designed to fit the volume of the hole or location 52 for receiving the radiopaque marker 40′. For example, the volumes can be substantially equal, or the wire volume can be slightly larger than the hole 52. This can enable complete or near complete filling of the hole 52.

The diameter of the wire can be approximately equal to, slightly larger, or slightly smaller than the diameter of the hole 52 for receiving the radiopaque marker 40′. Of course, other shapes besides, round wire and/or round spots for receiving the radiopaque marker can also be used. The length of wire can also correspond to the length of the location for receiving the radiopaque marker. In some embodiments, the volume of the amount of wire to be used can be approximately equal to or slightly larger than the volume of the location 52 for receiving the radiopaque marker, thus the diameter and/or the length may be equal to or slightly larger than that of the hole 52.

In some embodiments, a shape can be formed on the wire, such as on or near the end of the wire, to form a radiopaque marker 40″. For example, as shown in FIGS. 7A-B an end of a wire 42 can be advanced into a cavity 56 of a mold 54. Energy (E) may be applied to the wire 42 to more easily form the end of the wire into the desired shape. The energy can be in the form of heat, light, microwave, pressure, etc. Either or both of the wire 42 and the mold 54 may be moved relative to the other in forming the desired shape. For example, the mold 54 can be raised or the wire 42 advanced into the mold 54. As a further example, the wire 42 can be forced into a mold 54. In addition, energy such as heat energy may be imparted to the wire before forming.

The cavity 56 of the mold 54 can be one of any number of different shapes. For example, the cavity can be shaped to match hole 52 in the medical device 50. The volume and dimensions of the cavity can have characteristics similar to those already described for a wire based radiopaque marker, such as having a volume slightly greater than the hole 52. The cavity 56 is illustrated as a cylinder or disk, though other it could take many other forms. In addition, though the cavity is shown being larger than the wire outer diameter, in other embodiments, it can be smaller.

The design of the mold 54 can also allow for rapid fill-up of the cavity. It may act as a heat sink that can prevent material, such as gold, from sticking to walls of cavity. The cavity side walls can be at an angle or less than perpendicular, in particular so that the bottom of the cavity is smaller than the top of the cavity. This can enable ease of removal of the radiopaque marker 40″ from the cavity. This shape can also enable ease of insertion of the radiopaque marker 40″ into the hole 52 on the medical device 50.

With the wire 42 still attached to the formed shape of the radiopaque marker 40″, the radiopaque marker 40″ can be easily extracted from the mold 54 as shown in FIG. 7B. The radiopaque marker 40″ still connected to the wire 42 can then be positioned adjacent the desired location for attachment to a medical device. For example, FIG. 7C illustrates the radiopaque marker 40″ placed in the hole 52 on the medical device. Once at the desired location, the radiopaque marker 40″ can be separated from the wire 42. As illustrated, the cutting tool 38 can cut the wire 42 to separate the radiopaque marker 40″ from the rest of the wire 42. The cut can be made at the end of the formed share of the radiopaque marker 40″ or it may be spaced from there. In the illustrated embodiment, the radiopaque marker 40″ is cut at a location spaced away from the formed shape, leaving a tail 46. The formed wire can be positioned above the hole for attachment and then the wire can be cut such that the formed piece of wire is placed on, at, or in the hole. Energy, such as a force, can then be applied to the formed wire to attach it to the medical device as has been described previously.

In other embodiments, the radiopaque marker 40″ can be cut from the wire during the forming process. In such situations, a pick and place method could be employed to connect the radiopaque marker 40″ to the medical device.

FIGS. 8A-B show another method of forming a shape on a wire. In this method, energy such as a high-voltage electric discharge is applied to the end of the wire to form a ball. FIG. 8A illustrates a power source 58 and an electrode 60 that can discharge electricity. An arc of electricity can extend between the electrode 60 and the wire 42. The tip of the wire can form into a ball because of the surface tension of the molten metal. In some embodiments, a modified wire bonder machine can be used to form a ball as discussed above. These methods could also be employed with a mold, where the wire could be advanced into a mold after applying electric energy.

The size of wire and amount of electric discharge, among other factors, can be selected such that the ball or molded size achieves the desired size for the desired location 52, such as based on the size and volume of a hole 52 in a medical device 50. The ball 40″ can then be placed at, on or in the hole 52. The placement may be done with the same tool head that dispenses the wire or a separate tool head, such as a vacuum pickup tool head. In some embodiments, the wire and ball are cut after the ball has been placed at or in the insertion hole in the intravascular device. Energy, such as a force, is then applied to the ball as has been discussed to attach the ball to the device.

According to certain embodiments, a method of attaching a radiopaque marker to an intravascular device can comprise: advancing a wire such that a wire tip extends past a wire dispensing tool head; applying an electric discharge to the wire tip, thereby forming a ball; positioning the ball at a receiving hole in an intravascular device; cutting the wire; and applying a force on the ball to deform the ball such that the ball forms a radiopaque marker connected to the receiving hole.

In some embodiments, apply a force further comprises forming a first head at a first end of the marker and a second head at the opposite end. Some embodiments of the method may further comprise switching tool heads after cutting the wire, comprising switching from the dispensing tool head to a hammer tool head. In some embodiments, applying the force on the ball comprises applying the force with the hammer tool head.

Various cutting tools and methods will now be described. It will be understood that a wire based radiopaque marker can be cut from the wire spool at one of many different stages. For example, where a radiopaque marker ball or a molded radiopaque marker 40″ is formed, the wire would preferably be cut after formation. Of course, the wire could be cut before formation as well. As a further example, the ball 40″ may be cut before or after placement in the hole 52 of a medical device. The ball 40″ may also be cut before or after energy is applied to the ball to attach the ball to the hole 52.

The cut can be made in one of many different ways. For example, a blade, scissors, or pinchers can be used. In another example, the wire can be broken by two opposing forces, such as one pressing down on the radiopaque marker and the other pulling away therefrom. As shown above, a pair of blades can cut the wire a set distance above the ball.

In the embodiments shown in FIGS. 6B-C and 7A-C, the cutting tool 38 includes two opposing blades, each extending at an angle from an arm member. The arm members can be hinged to move closer together to create the cutting motion. The blades can be shaped to control the shape of the wire tip that is cut. For example, the point or angles on the tip can be set at particular angles to get a desired surface shape on the radiopaque marker.

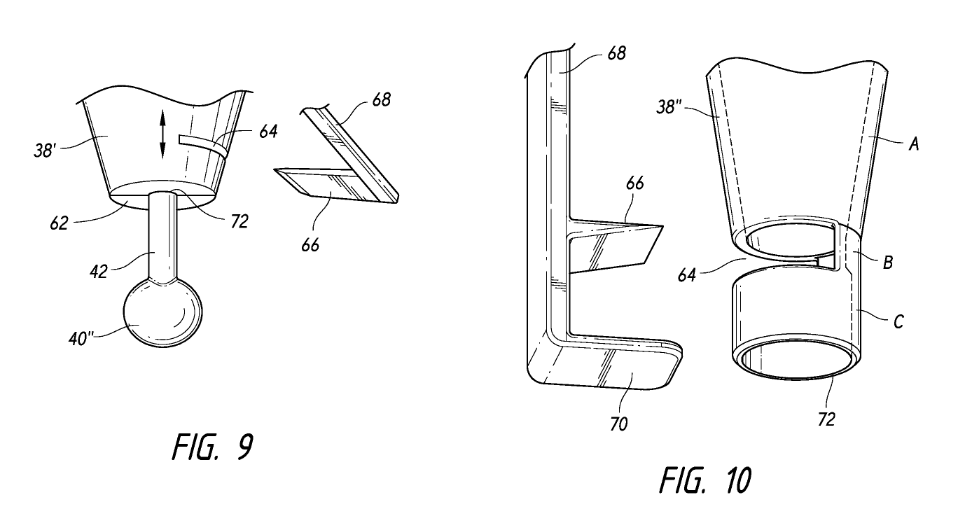

Looking now to FIG. 9, another embodiment of a tool head with cutting tool 38′ is shown. The tool head may be used to control the cut of the wire. For example, the cut can be made at or against the bottom 62 of the tool head. For example, a blade 66 extending from an arm 68 can slide across the bottom 62 of the tool head to cut the wire 42. Alternatively, the tool head can include one or more slots 64 that allow the cut to be made through the tool head. For example, a blade 66 or a laser beam can pass through or into the tool head at the slot to cut the wire 42.

The tool head can also help the wire maintain its shape after it is cut. For example, the exit hole 72 on the tool head 38 can be shaped to be substantially similar to the shape of the wire 42. The exit hole 72 is shown to be slightly larger than the diameter of the wire 42. Thus, the hole size can be large enough to not create a large amount of friction, and yet close enough to the size of the wire so to prevent or reduce the likelihood that the cutting action will force the wire to take on a new shape. Thus, the tool head can minimize the impact of cutting the wire on the shape of the wire for the next radiopaque marker. This can be the case whether or not the wire will be further formed into a ball or molded.

It will be understood that the tool head 38′ can move as indicated by the two way arrow. Also, the tool head 38′ can control the amount of wire that extends past the tool head. In the case of ball formation, the ball 40″ may be moved upwards back towards the tool head to provide better control for ball placement.

Looking now to FIG. 10, still another embodiment of a tool head with cutting tool 38″ is shown. In this embodiment, the tool head 38″ is made to both cut the wire and hold the radiopaque marker that is formed by the cutting. An arm 68 can connect a blade 66 to cut the wire and a retainer 70 to be positioned below the exit hole 72. Thus, as the blade cuts the wire, the retainer 70 will be in position to prevent the radiopaque marker from falling out of the tool head exit hole 72. The tool head can then be accurately positioned at a desired location 52, before releasing the radiopaque marker by moving the retainer 70 from covering the exit hole 72. This will allow the radiopaque marker to fall into or be placed upon the desired location 52. In some embodiments, the tool head may also be moved upwards to fully release the radiopaque marker from the tool head.

The tool head is shown with three sections A, B & C. The first section can be for loading the wire prior to performing the cut. For example, the first section A can have a funnel or tapered inner cross section. This can permit the wire to be properly positioned at the slot 64 so that the wire can be precisely cut to form the radiopaque marker. This cross-sectional shape can also help to reduce friction within the tool head.

The slot 64 can be located in section B. Section B in some embodiments may extend slightly above and below the slot 64. Section B may provide one or more features in addition to having the cutting slot 64. For example, section B can provide a backstop for the cutting blade 66. In addition, section B may include a softer material, to absorb some of the impact from the cutting blade 66. This softer material may also help prevent the wire from taking on another shape as it is cut.

In some embodiments, section B can be shaped to correspond with a desired shape of the radiopaque marker. Thus, during cutting, when the wire is pressed into contact with the section B, any force that might cause the wire to deform can be channeled to the desired shape of the radiopaque marker. In some embodiments, this shape can correspond with the initial outer diameter of the wire. The section B may cup the wire and hold it in a desired manner.

The cross section of the open space in section C can be slightly larger than the final cross section of the open space of section A. This can allow the radiopaque marker to freely fall into section C. Though the cross section of the open space in section C needs to be larger than the width or diameter of the radiopaque marker, it is preferably less than the height of the radiopaque marker. This will help prevent the radiopaque marker from being able to become disorientated, such as becoming sideways.

In some embodiments, a method of attaching a radiopaque marker to an intravascular device can comprise: positioning an end of a wire in a wire dispensing tool head above a receiving hole in an intravascular device; placing the end of the wire on or in the receiving hole; cutting the wire; and applying a force on the cut wire to deform the cut wire such that the cut wire forms a radiopaque marker connected to the receiving hole.

Some embodiments may further comprise molding the end of the wire into a desired shape before cutting the wire. Molding the end can comprise applying an electric discharge to the end, thereby forming a ball. Molding the end can comprise forcing the end into a mold. Apply a force may further comprise forming a first head at a first end of the marker and a second head at the opposite end.

Some embodiments may further comprise switching tool heads after cutting the wire, comprising switching from the dispensing tool head to a hammer tool head. Applying the force on the cut wire can comprise applying the force with the hammer tool head.

According to some embodiments, a system can be used for attaching a radiopaque marker to an intravascular device. The system can include a wire dispensing tool, a cutting tool, and a hammer tool. The wire dispensing tool can be configured to precisely position an end of a wire at a receiving hole in an intravascular device. The cutting tool can be configured to cut off the end of the wire after it is positioned at the receiving hole. The hammer tool can be configured to apply a force on the cut wire end to deform the wire end such that the wire end connects to the receiving hole.

The system can include any of the other features described herein. For example the system may further include a mold configured to shape the end of the wire into a desired shape before cutting the wire. The system can further include a power source and an electrode configured to apply an electric discharge to the wire end to thereby form a ball.

In some embodiments, the wire dispensing tool and the cutting tool are part of the same tool head. The wire dispensing tool can have a slot to receive the cutting tool to cut the wire end. A retainer can be configured to hold the cut wire end within the wire dispensing tool after it has been cut.

Although this invention has been disclosed in the context of certain preferred embodiments and examples, it will be understood by those skilled in the art that the present invention extends beyond the specifically disclosed embodiments to other alternative embodiments and/or uses of the invention and obvious modifications and equivalents thereof. In addition, while a number of variations of the invention have been shown and described in detail, other modifications, which are within the scope of this invention, will be readily apparent to those of skill in the art based upon this disclosure. It is also contemplated that various combinations or sub-combinations of the specific features and aspects of the embodiments may be made and still fall within the scope of the invention. Accordingly, it should be understood that various features and aspects of the disclosed embodiments can be combined with or substituted for one another in order to form varying modes of the disclosed invention.

What is claimed is:

1. A method of attaching a radiopaque marker to an intravascular device comprises:
positioning an end of a wire in a wire dispensing tool head above a receiving hole in an intravascular device;
placing the end of the wire on or in the receiving hole;
cutting the wire;
applying a force on the cut wire to deform the cut wire such that the cut wire connects to the receiving hole.

2. The method of claim 1, further comprising molding the end of the wire into a desired shape before cutting the wire.

3. The method of claim 2, wherein molding the end comprises applying an electric discharge to the end, thereby forming a ball.

4. The method of claim 1, wherein apply a force further comprises forming a first head at a first end of the marker and a second head at the opposite end.

5. The method of claim 1, further comprising switching tool heads on an assembly system after cutting the wire, comprising switching from the dispensing tool head to a hammer tool head.

6. The method of claim 5, wherein applying the force on the cut wire comprises applying the force with the hammer tool head.

7. A system for attaching a radiopaque marker to an intravascular device comprising:
a wire dispensing tool configured to precisely position an end of a wire at a receiving hole in an intravascular device;
a cutting tool configured to cut off the end of the wire after it is positioned at the receiving hole; and
a hammer tool configured to apply a force on the cut wire end to deform the wire end such that the wire end connects to the receiving hole.

8. The system of claim 7, further comprising a mold configured to shape the end of the wire into a desired shape before cutting the wire.

9. The system of claim 7, further comprising a power source and an electrode configured to apply an electric discharge to the wire end to thereby form a ball.

10. The system of claim 7, wherein the wire dispensing tool and the cutting tool are part of the same tool head.

11. The system of claim 10, wherein the wire dispensing tool comprises a slot to receive the cutting tool to cut the wire end.

12. The system of claim 11, further comprising a retainer configured to hold the cut wire end within the wire dispensing tool after it has been cut.

13. A method of attaching a radiopaque marker to an intravascular device comprises:
advancing a wire such that a wire tip extends past a wire dispensing tool head;
applying an electric discharge to the wire tip, thereby forming a ball;
positioning the ball at a receiving hole in an intravascular device;
cutting the wire;
applying a force on the ball to deform the ball such that the ball forms a radiopaque marker connected to the receiving hole.

14. The method of claim 13, wherein apply a force further comprises forming a first head at a first end of the marker and a second head at the opposite end.

15. The method of claim 13, further comprising switching tool heads after cutting the wire, comprising switching from the dispensing tool head to a hammer tool head.

16. The method of claim 15, wherein applying the force on the ball comprises applying the force with the hammer tool head.

17. A method of attaching a radiopaque marker to an intravascular device comprises:
applying suction to a radiopaque marker;
positioning the radiopaque marker above a receiving hole in an intravascular device;
releasing suction from the radiopaque marker such that the radiopaque marker is placed on the receiving hole;
applying a force on the radiopaque marker to deform the radiopaque marker such that the radiopaque marker connects to the receiving hole, a first head forming at a first end of the radiopaque marker and a second head forming at the opposite end.

18. The method of claim 17, wherein applying suction comprises applying suction with a vacuum pickup tool head on an assembly system and positioning the radiopaque marker comprises positioning the radiopaque marker with the vacuum pickup tool head.

19. The method of claim 18, further comprising switching tool heads on the assembly system after releasing suction, comprising switching from the vacuum pickup tool head to a hammer tool head.

20. The method of claim 19, wherein applying the force on the radiopaque marker comprises applying the force exceeding 0.1N with the hammer tool head.
